(19) United States
(12) Patent Application Publication
(10) Pub. No.: US 2007/0032506 A1
(43) Pub. Date: Feb. 8, 2007

(54) CRYSTALLINE FORMS OF (2R-TRANS)-6-CHLORO-5[[4-[(4-FLUOROPHENYL)METHYL]-2,5-DIMETHYL-1-PIPERAZINYL]CARBONYL]-N,N, 1-TRIMETHYL-ALPHA-OXO-1H-INDOLE-3-ACETAMIDE MONOHYDROCHLORIDE

(76) Inventors: Peter Giannousis, Pacifica, CA (US); Kathleen M. Lee, Palo Alto, CA (US); Sundeep Dugar, San Jose, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/427,768

(22) Filed: Jun. 29, 2006

Related U.S. Application Data
(60) Provisional application No. 60/696,181, filed on Jul. 2, 2005.

(51) Int. Cl. A61K 31/496; C07D 403/02

(52) U.S. Cl. 514/254.09; 544/373

(57) ABSTRACT

The present invention relates to novel crystalline forms of (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monochloride, methods for their preparation, and pharmaceutical compositions comprising the novel pseudopolymorphs.

### RELATED APPLICATIONS

This application claims the benefit of priority of U.S. Provisional Patent Application No. 60/696,181 filed Jul. 2, 2005, which is hereby incorporated by reference in its entirety.

### TECHNICAL FIELD

The present invention relates to novel crystalline pseudopolymorphs of (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monohydrochloride, methods for their preparation, and pharmaceutical compositions comprising the novel pseudopolymorphs.

### BACKGROUND OF THE INVENTION

The compound (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monohydrochloride is an agent that can be used to treat a variety of disorders such as multiple myeloma, metastic cancers and bone disease, psoriasis, rheumatoid arthritis and other inflammatory related disorders. Its utility and preparation are described in U.S. Pat. No. 6,867,209, issued Mar. 15, 2005, which is hereby incorporated by reference in its entirety.

With respect to both the Active Pharmaceutical Ingredient (API) to be formulated into a pharmaceutical product or the product itself, manufacturing processes are required that yield products which are consistent and stable. Strictly regulated by high standards mandated by the USFDA and analogous agencies throughout the world, pharmaceuticals must meet tight specifications to ensure compound safety and efficacy.

Physiochemical properties of a compound can influence its solid state characteristics. In this regard, one challenge is making a product that will behave consistently under different conditions. Having a homogenous product helps to achieve such consistency. Such a property is desired in that it typically will result in lower variability with respect to solubility and concentration, each of which can have a potential effect on the pharmacokinetic and pharmacodynamic properties of the pharmaceutical product.

Another challenge for making a viable pharmaceutical product is producing a product at commercial scale under varying and unavoidable conditions. Commercial manufacturing facilities are typically maintained under humidity conditions in the range of 40 to 75% RH. In this context, it is desirable to have a form of (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide that can be maintained in a stable hydration state under standard and typical storage conditions.

It has now been unexpectedly discovered that the aforementioned compound can be prepared in a new and stable crystalline state which is termed Type II.

### SUMMARY OF THE INVENTION

The present invention is directed to novel crystalline forms of the compound (2R-trans)-6-chloro-5-[[4-[(4-fluorophenyl)methyl]-2,5-dimethyl-1-piperazinyl]carbonyl]trimethyl-alpha-oxo-1H-indole-3-acetamide monohydrochloride.

In one embodiment, the invention is directed to Type I, a tetrahydrate form of the compound (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monohydrochloride.

In a preferred embodiment, the invention is directed to Type II, a stable dihydrate crystalline form of the compound (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monohydrochloride.

In another embodiment, the invention is directed to a method for producing Type II.

In yet another embodiment, the invention is directed to a pharmaceutical composition comprising Type II.

### DESCRIPTION OF THE FIGURES

FIG. 1 is the molecular structure of the compound (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monohydrochloride.

FIG. 2 is an X-ray Powder Diffraction (XRPD) tracing of the compound in the present invention.

FIG. 3 is an X-ray Powder Diffraction (XRPD) tracing of the compound in varying forms. The upper tracing is for the Type II crystal that was crystallized from isopropanol and thereafter used to solve the dihydrate single crystal structure, the middle tracing is for Compound in hygroscopically stable Type II as it is produced in the processes provided in the Examples, and the lower tracing is the calculated XRPD pattern based upon the determined crystal structure of the tetrahydrated Compound (Type I).

FIG. 4 is DSC thermogram of the Compound. Applied heating rate 20° C./minute. Tₒₙₛₑₜ=40° C., Tₚₑₐₖ=63.6° C.; Tₒₙₛₑₜ=96.1° C., Tₚₑₐₖ=133.0° C.; Tₒₙₛₑₜ=187.3° C.

FIG. 5 is a thermogram of (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monohydrochloride. Applied heating rate 20° C./min. Mass losses of 3.3% and 2% are detected in the temperature intervals 30-120 and 120-190° C., respectively.

FIG. 6 shows the molecular structure for the dehydrate Type II form of the compound.

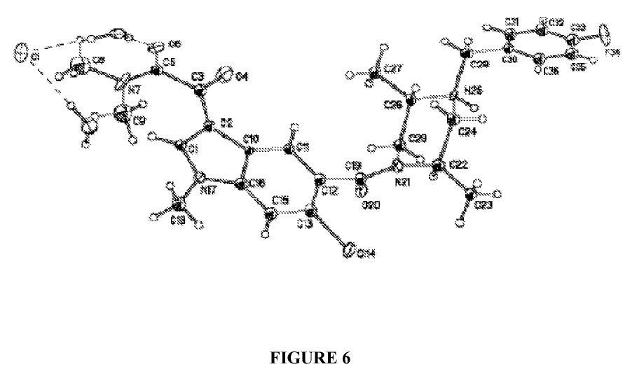

### DETAILED DESCRIPTION OF THE INVENTION

Definitions:

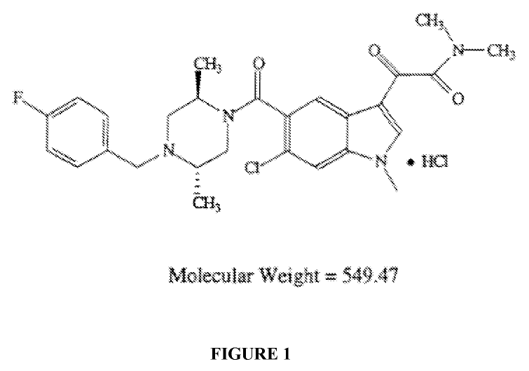

Compound means (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monohydrochloride in crystalline form as provided herein. The molecular structure of Compound is provided in FIG. 1.

Material means Type II product.

Type I means (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monohydrochloride in a tetrahydrate crystalline structure.

Type II means (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monohydrochloride in a dihydrate crystalline structure.

As previously mentioned above, the compound (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide monohydrochloride and methods for its preparation are described in U.S. Pat. No. 6,867,209, issued Mar. 15, 2005. Although the patent describes the Compound as it can be provided in any form, it does not teach how to produce the Compound in a substantially pure crystalline state of either Type I or Type II.

In general, the present invention involves transforming the free base of (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide into the monohydrochloride form in the presence of water under controlled solutions. Essentially, the free base is converted to the hydrochloride salt by reaction with hydrochloric acid in a THF aqueous solution. Under a vacuum, the Material is slowly dried until the desired crystallization is achieved. The resulting material is a white powder that exhibits the XRPD diffraction properties as provided in Table 1 below.

TABLE 1
X-ray Powder Diffraction (using Copper Kα radiation)
2θ angles and Relative Intensities of Major Peaks Observed

| 2 Theta | D space | Relative |
|---|---|---|
| Angle (°) | Å | Intensity |
| 19.316 | 4.592 | 100.0 |
| 22.024 | 4.032 | 59.0 |
| 25.428 | 3.450 | 54.1 |
| 16.950 | 5.227 | 46.5 |
| 10.771 | 8.207 | 43.9 |
| 29.836 | 2.992 | 43.4 |
| 23.123 | 3.843 | 39.3 |
| 13.279 | 6.662 | 38.0 |
| 13.489 | 6.559 | 32.7 |
| 18.124 | 4.891 | 31.0 |
| 19.738 | 4.494 | 30.6 |
| 6.738 | 13.107 | 30.4 |
| 24.086 | 3.692 | 30.0 |
| 27.072 | 3.291 | 29.1 |
| 12.460 | 7.098 | 28.2 |
| 22.901 | 3.880 | 27.9 |
| 16.706 | 5.302 | 22.3 |
| 23.600 | 3.767 | 20.1 |
| 29.419 | 3.034 | 19.1 |
| 20.089 | 4.417 | 18.3 |
| 24.412 | 3.643 | 17.5 |
| 32.753 | 2.732 | 15.1 |
| 30.282 | 2.949 | 15.1 |
| 15.046 | 5.884 | 14.4 |
| 20.979 | 4.231 | 13.4 |

In an alternative embodiment, the compound can be crystallized through the use of a seed crystal. The free base of the Compound is suspended in a mixture of isopropanol/water, and acidified with concentrated aq. HCl. The resulting solution is filtered, and MTBE is added, followed by seed crystals of the Type I (Dihydrate). After about 20 min, additional MTBE is added, and after 3-6 hours, the product is isolated by filtration, rinsed with a mixture of MTBE/isopropanol, and dried to 4-6% water under reduced pressure.

Formulations and Dosing

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 0. 1 to about 95 percent active ingredient. Suitable solid carriers are known in the art, e.g., microcrystalline cellulose, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), The Science and Practice of Pharmacy, 20.sup.th Edition, Lippincott Williams & Wilkins, Baltimore, Md., (2000).

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations that are intended to be converted, shortly before use, to liquid form preparations for either oral or parenteral administration. Such liquid forms include solutions, suspensions and emulsions.

The Compound of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the Compound is administered orally. Preferably, the pharmaceutical preparation is in a unit dosage form. In such form, the preparation is subdivided into suitably sized unit doses containing appropriate quantities of the active component, e.g., an effective amount to achieve the desired purpose.

The amount and frequency of administration of the Compound will be regulated according to the judgment of the attending clinician considering such factors as age, condition and size of the patient as well as severity of the symptoms being treated.

Further embodiments of the invention encompass the administration of the Compound along with at least one additional therapeutically effective agent. The contemplated additional therapeutically effective agent is one that differs in either atomic make up or arrangement from the Compound. Therapeutically effective agents that can be used in combination with the novel compounds of this invention include drugs that are known and used in the treatment of inflammation, rheumatism, asthma, glomerulonephritis, osteoporosis, neuropathy and/or malignant tumors, angiogenesis related disorders, cancer, disorders of the liver, kidney and lung, melanoma, renal disease, chronic airways disease, bladder inflammation, neurodegenerative and/or neurotoxic diseases, conditions, or injuries. Further examples of therapeutically effective agents which may be administered in combination with the Compound include resistance factors for tumor cells towards chemotherapy.

Further embodiments of the invention encompass the administration of the Compound along with more than one additional therapeutically effective agent. In these embodiments, the additional therapeutically effective agent may or may not be commonly used in the treatment of the same condition.

When the invention comprises a combination of the Compound and one or more other therapeutically effective agents, the two or more active components may be co-administered simultaneously or sequentially, or a single pharmaceutical composition comprising the Compound and the other therapeutically effective agent(s) in a pharmaceutically acceptable carrier can be administered. The components of the combination can be administered individually or together in any conventional dosage form such as capsule, tablet, powder, cachet, suspension, solution, suppository, nasal spray, etc. The dosage of the other therapeutically active agent(s) can be determined from published material, and may range from 1 to 1000 mg per dose.

Pharmaceutically-acceptable excipients or carriers comprise flavoring agents, pharmaceutical-grade dyes or pigments, solvents, co-solvents, buffer systems, surfactants, preservatives, sweetener agents, viscosity agents, fillers, lubricants, glidants, disintegrants, binders and resins.

Conventional flavoring agents can be used, such as those described in Remington's Pharmaceutical Sciences, 18.sup.th Ed., Mack Publishing Co., 1288-1300 (1990), which is incorporated by reference herein. The pharmaceutical compositions of the invention generally comprise from 0% to about 2% of flavoring agent(s).

Conventional dyes and/or pigments can also be used, such as those described in the Handbook of Pharmaceutical Excipients, by the American Pharmaceutical Association & the Pharmaceutical Society of Great Britain, 81-90 (1986), which is incorporated by reference herein. The pharmaceutical compositions of the invention generally comprise from 0% to about 2% of dye(s) and/or pigment(s).

The pharmaceutical compositions of the invention generally comprise from about 0.1% to about 99.9% of solvent(s). A preferred solvent is water. Preferred co-solvents comprise ethanol, glycerin, propylene glycol, polyethylene glycol, and the like. The pharmaceutical compositions of the invention can comprise from 0% to about 50% of co-solvent(s).

Preferred buffer systems comprise acetic, boric, carbonic, phosphoric, succinic, malic, tartaric, citric, acetic, benzoic, lactic, glyceric, gluconic, glutaric and glutamic acids and their sodium, potassium and ammonium salts. Particularly preferred buffers are phosphoric, tartaric, citric and acetic acids and salts thereof. The pharmaceutical compositions of the invention generally comprise from 0% to about 5% of buffer(s).

Preferred surfactants comprise polyoxyethylene sorbitan fatty acid esters, polyoxyethylene monoalkyl ethers, sucrose monoesters and lanolin esters and ethers, alkyl sulfate salts and sodium, potassium and ammonium salts of fatty acids. The pharmaceutical compositions of the invention generally comprise from 0% to about 2% of surfactant(s).

Preferred preservatives comprise phenol, alkyl esters of parahydroxybenzoic acid, o-phenylphenol benzoic acid and salts thereof, boric acid and salts thereof, sorbic acid and salts thereof, chlorobutanol, benzyl alcohol, thimerosal, phenylmercuric acetate and nitrate, nitromersol, benzalkonium chloride, cetylpyridinium chloride, methyl paraben and propyl paraben. Particularly preferred preservatives are the salts of benzoic acid, cetylpyridinium chloride, methyl paraben and propyl paraben. The pharmaceutical compositions of the invention generally comprise from 0% to about 2% of preservative(s).

Preferred sweeteners comprise sucrose, glucose, saccharin, sorbitol, mannitol and aspartame. Particularly preferred sweeteners are sucrose and saccharin. Pharmaceutical compositions of the invention generally comprise from 0% to about 5% of sweetener(s).

Preferred viscosity agents comprise methylcellulose, sodium carboxymethylcellulose, hydroxypropyl-methylcellulose, hydroxypropylcellulose, sodium alginate, carbomer, povidone, acacia, guar gum, xanthan gum and tragacanth. Particularly preferred viscosity agents are methylcellulose, carbomer, xanthan gum, guar gum, povidone, sodium carboxymethylcellulose, and magnesium aluminum silicate. Pharmaceutical compositions of the invention generally comprise from 0% to about 5% of viscosity agent(s).

Preferred fillers comprise lactose, mannitol, sorbitol, tribasic calcium phosphate, diabasic calcium phosphate, compressible sugar, starch, calcium sulfate, dextro and microcrystalline cellulose. Pharmaceutical compositions of the invention generally comprise from 0% to about 90% of filler(s).

Preferred lubricants/glidants comprise magnesium stearate, stearic acid and talc. Pharmaceutical compositions of the invention generally comprise from 0% to 7%, preferably, from about 1% to about 5%, of lubricant(s)/glidant(s).

Preferred disintegrants comprise starch, sodium starch glycolate, crospovidone and croscarmelose sodium and microcrystalline cellulose. Pharmaceutical compositions of the invention generally comprise from 0% to about 20%, preferably, from about 4% to about 15%, of disintegrant(s).

Preferred binders comprise acacia, tragacanth, hydroxypropylcellulose, pregelatinized starch, gelatin, povidone, hydroxypropylcellulose, hydroxypropylmethylcellulose, methylcellulose, sugar solutions, such as sucrose and sorbitol, and ethylcellulose. Pharmaceutical compositions of the invention generally comprise from 0% to about 12%, preferably, from about 1% to about 10%, of binder(s).

Additional agents known to a skilled formulator may be combined with the inventive compounds to create a single dosage form. Alternatively, additional agents may be separately administered to a mammal as part of a multiple dosage form.

A pharmaceutical composition typically comprises from about 0. 1% to about 99.9% (by weight or volume, preferably, w/w) of active ingredient, preferably, from about 5% to about 95%, more preferably, from about 20% to about 80%. For preparing pharmaceutical compositions comprising the Compound, inert, pharmaceutically acceptable excipients or carriers can be either solid or liquid. Solid Type preparations comprise powders, tablets, dispersible granules, capsules, cachets and suppositories. Suitable solid excipients or carriers are known in the art, for example, magnesium carbonate, magnesium stearate, talc, sugar and lactose. Tablets, powders, cachets and capsules can be used as solid dosage Types suitable for oral administration. Examples of pharmaceutically-acceptable excipients or carriers and methods of manufacture for various compositions may be found in Remington's Pharmaceutical Sciences, 18.sup.th Ed., Mack Publishing Co. (1990), which is incorporated in its entirety by reference herein.

Liquid Type preparations comprise solutions, suspensions and emulsions. Common liquid type preparations comprise water and water-propylene glycol solutions for parenteral injection or addition of sweeteners and opacifiers for oral solutions, suspensions and emulsions. Liquid type preparations may also comprise solutions for intranasal administration.

The quantity of the Compound in a unit dose of preparation may be varied or adjusted from about 0.01 mg to about 4,000 mg, preferably, from about 0.02 mg to about 2,000 mg, more preferably, from about 0.03 mg to about 1,000 mg, even more preferably, from about 0.04 mg to about 500 mg, and most preferably, from about 0.05 mg to about 250 mg, according to the particular application. A typical recommended daily dosage regimen for oral administration can range from about 0.02 mg to about 2,000 mg/day, in two to four divided doses. For convenience, the total daily dosage may be divided and administered in portions during the day as required. Typically, pharmaceutical compositions of the invention will be administered from about 1 time per day to about 5 times per day, or alternatively, as a continuous infusion. Such administration can be used as a chronic or acute therapy.

The pharmaceutically-acceptable excipients or carriers employed in conjunction with the compounds of the present invention are used at a concentration sufficient to provide a practical size to dosage relationship. The pharmaceutically-acceptable excipients or carriers, in total, can comprise from about 0.1% to about 99.9% (by weight or volume, preferably, by w/w) of the pharmaceutical compositions of the invention, preferably, from about 5% to about 95% by weight, more preferably, from about 20% to about 80% by weight.

Specific dosage and treatment regimens for any particular patient may be varied and will depend upon a variety of factors, including the activity of the specific compound employed, the age, body weight, general health status, sex and diet of the patient, the time of administration, the rate of excretion, the specific drug combination, the severity and course of the symptoms being treated, the patient's disposition to the condition being treated and the judgment of the treating physician. Determination of the proper dosage regimen for a particular situation is within the skill of the art. The amount and frequency of the administration of the Compound, or the pharmaceutical compositions thereof, may be regulated according to the judgment of the attending clinician, based on the factors recited above. As a skilled artisan will appreciate, lower or higher doses than those recited above may be required.

For instance, it is often the case that a proper dosage level is based on the weight of the patient. For example, dosage levels of between about 0.01 mg/kg and about 100 mg/kg of body weight per day, preferably, between about 0.5 mg/kg and about 75 mg/kg of body weight per day, and more preferably, between about 1 mg/kg and about 50 mg/kg of body weight per day, of the inventive compound(s), and compositions described herein, are therapeutically useful for the treatment of a variety of biological disorders. Between two patients of differing weights, a higher dosage will be used for the heavier patient, all other things being equal.

Crystalline Purity

Preferably, the Compound is in the form of Type II, substantially free of chemical impurities (e.g., by-products generated during the preparation of Compound) and of associate crystalline forms. “Substantially free” of chemical impurities for the purposes of this invention means less than or equal to about 5% w/w of chemical impurities, preferably, less than or equal to about 3% w/w of chemical impurities, more preferably, less than or equal to about 2% w/w of chemical impurities, and even more preferably, less than or equal to about 1% w/w of chemical impurities. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

The following examples illustrate the present invention in more detail. They are, however, not intended to limit its scope in any manner.

### EXAMPLES

### Example 1

An amount of 82.0 kg of (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide, freebase was dissolved in 605 kg of THF, filtered, and the volume was reduced to 170-183 L by vacuum distillation. Approx. 1.0 equivalent of 18% aqueous HCl was added, followed by about 330 kg of water, and the mixture was heated to about 35° C. The volume was reduced to about 415 L by vacuum distillation, at which point (or earlier) crystallization occurred. The slurry was stirred at 20-25° C. for at least 30 minutes, cooled to 0-5° C., stirred for at least 2 hours. The product was isolated by filtration, washed sequentially with water (47 kg), and isopropyl acetate (232-326 kg). The wet cake was dried under vacuum, with the dryerjacket temperature at 50° for about 10 hours, then at batch temperature of 20 to 25° C. until the water content reached 5-7% as measured by Karl-Fischer titration, which is the hydrochloride dihydrate. The resulting free-flowing powder was discharged to give about 78 kg (89% yield). The XRPD pattern of this material was identical to the XPRD of the solved X-ray crystal structure of the monohydrochloride dihydrate.

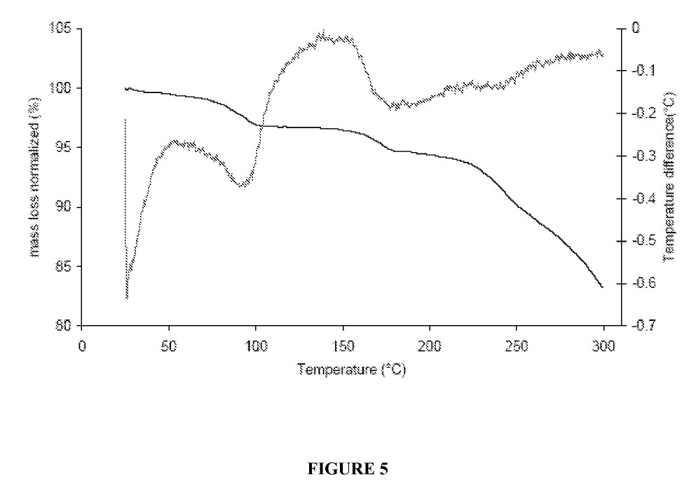

The material was subsequently evaluated by thermo gravimetric analysis (TGA). Its mass loss due to solvent or water loss from the crystals is provided in FIG. 5. During heating of a material sample in a TGA/SDTA851e device (Mettler-Toledo GmbH, Switzerland), the weight of the sample was monitored and yielded a weight v. temperature curve. The TGA/SDTA851e device was calibrated for temperature with indium and aluminum. Samples were weighed in 100 μl aluminum crucibles and heated in the TGA from 25 to 300° C. with a heating rate of 20° C. min⁻¹. Dry Nitrogen gas was used for purging.

In the TGA trace, a mass loss in two steps of 3.3% and 2% was observed in the temperature intervals of 30 to 120° C. and 120 to 190° C., respectively. This implies a loss of approximately two water molecules indicating a di-hydrate (Type II) form of the Compound.

The melting properties of the same material were also obtained by differential scanning calorimetry (DSC) thermograms. This was accomplished with a DSC822e device (Mettler-Toledo GmbH, Switzerland). The device was calibrated for temperature and enthalpy with a small piece of indium (melting point=156.6° C.; ΔHf=28.45 J.g⁻¹). The samples were sealed in standard 40 μl aluminum pans and heated in the DSC from 25 to 300° C. with a heating rate of 20° C. min⁻¹. Dry Nitrogen gas was used to purge the DSC equipment during measurement at a flow rate of 50 ml min⁻¹.

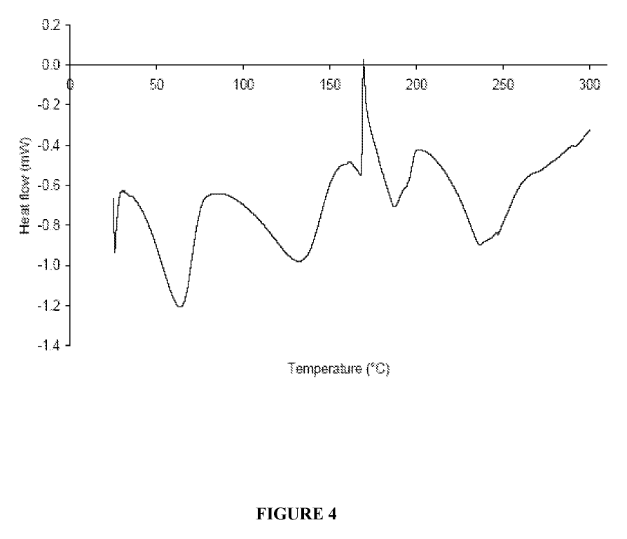

In the DSC thermogram (see FIG. 4), the material is characterized by three main broad endothermic events at 63.6, 133.0 and 187.3° C. These events tend to correlate with the TGA trace.

### Example 2

### Recrystallization and Characterization of the Compound

The Material obtained from example 1 was recrystalized in both water and isopropanol. For the recrystallization in water, 30 mg of the Material was suspended in 200 μL of water. The mixture was heated up to 60° C. in order to obtain a complete dissolution and slowly cooled to room temperature. While the suspension was left for several days at room temperature, small crystals began to grow. The solution was heated again to 40° C. to reach partial dissolution and slowly cooled to room temperature. The process was repeated until large colorless needle shaped crystals were obtained for analysis.

A second procedure was followed to recrystallize the Material from isopropanol. An amount of 45 mg of the Material was suspended in 300 μl of isopropanol. The mixture was heated close to the solvent boiling point such that a clear solution was obtained. The solution was then cooled slowly to 30° C. and very thin needle shaped crystals appeared. This procedure was repeated several times (heated to 50° C. and cooled to 30° C. with a cooling rate of 1°/hr). A single crystal data collection at 120K was conducted.

The material from Example 1 and the two sets of recrystallized Material as provided above were characterized by Powder X-ray diffraction (XRPD) patterns. XRPD patterns were obtained using a proprietary high throughput setup (Avantium's® T2 high throughput XRPD setup). Plates were mounted on a Bruker GADDS diffractometer that was equipped with a Hi-Star area detector. The XRPD platform was calibrated using Silver Behenate for the long d-spacings and Corundum for the short d-spacings. Data collection was carried out at room temperature using monochromatic CuK_{α} radiation in the region of 2θ between 1.5 and 41.5°. The diffraction pattern was collected in two 2θ ranges (1.5≦20≦19.5° for the first frame, and 21.5≦20≦41.5° for the second frame) with an exposure time between 90 and 180 seconds for each frame. No background subtraction or curve smoothing was applied to the XRPD patterns.

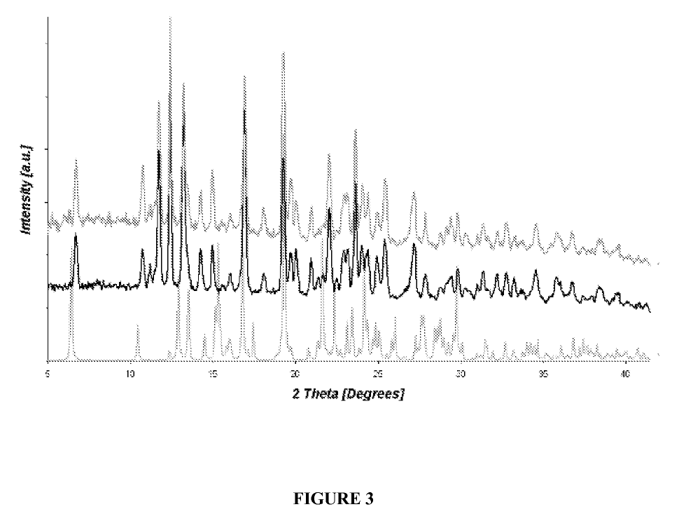

The resulting XRPD patterns are shown in FIG. 3. The upper tracing resulted from the material that was recrystallized with isopropanol, the middle tracing resulted from the material as provided from Example 1, and the lower tracing resulted from the material that was recrystallized with water. The upper and middle tracings are substantially similar indicating that the material recrystallized with isopropanol is Compound in the form of a dihydrate. The lower tracing, which was generated by recrystallization with water, has some remarkable differences from the middle tracing. It has been solved that the material recrystallized with water is in a tetrahydrate Type I form.

Under conditions of high humidity, it is likely that the Compound exists in a more hydrated state (Type I). Under standard conditions however, the Compound finds stability in the Type II form.

### Example 3

In an earlier study, Material was recrystallized similar to Example 2 above. For example, approximately 200 mg of material was mixed with ˜15 to 20 mL of isopropanol in a 100 mL beaker. The material was dissolved by stirring on a stir plate. It was removed from the stir plate, covered with a Kimwipe and allowed to evaporate at room temperature. The material recrystallized from isopropanol and water evaporated to an agglomerated powder. The recrystallized product was evaluated by another XRPD analysis. XRPD patterns for samples of this Material were obtained either on a Scintag XDS 2000 θ/θ diffractometer operating with copper radiation at 45 kV and 40 mA, using a Kevex Psi Peltier-cooled silicon detector, or a Scintag X₂ θ/θ diffractometer operating with copper radiation at 45 kV and 40 mA using a Scintag Scintillation detector. Source slits of 2 and 4 mm, and detector slits of 0.5 and 0.3 mm, (or 0.5 and 0.2 mm for the X₂ unit) were used for data collection.

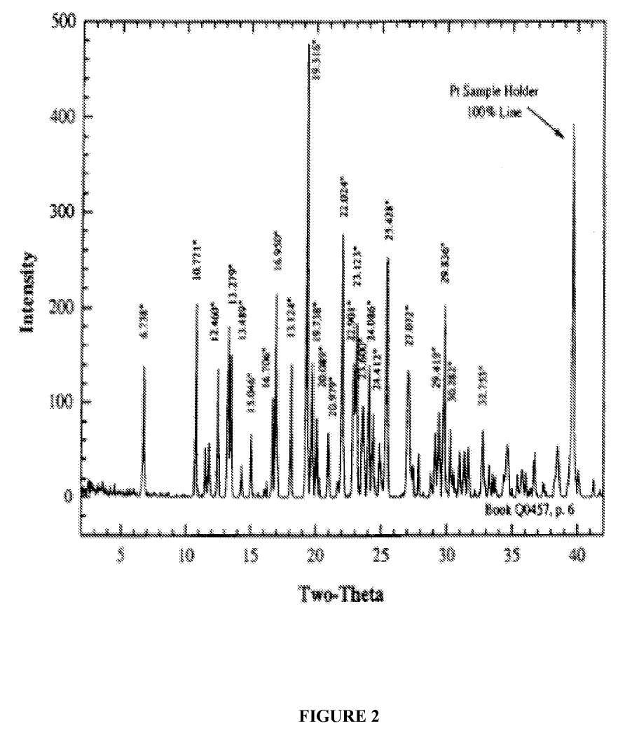

The results of the XRPD analysis are shown both in Table I above and in FIG. 2. Dynamic Vapor Sorption analysis of this same materials was also done. The results demonstrated that the Compound rapidly gains water weight to about 6-7%, and that the water content remains at 6-7% over a humidity range from ˜21-70% Relative Humidity, suggesting hydration of the crystalline monochloride. (2 moles of water per molecule of the Compound would be 6.15% water).

Other than as shown in the operating examples or as otherwise indicated, all numbers used in the specification and claims expressing quantities of ingredients, reaction conditions, and so forth, are understood as being modified in all instances by the term “about.”

The foregoing is merely illustrative of the invention and is not intended to limit the invention to the disclosed embodiments. Variations and changes which are obvious to one skilled in the art are intended to be within the scope and nature of the invention which are defined in the appended claims.

What is claimed is:

1. A crystal comprising the compound of the formula

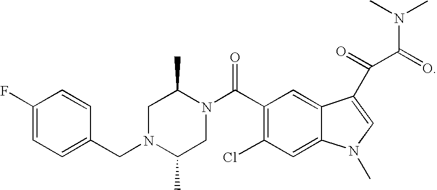

2. The crystal of claim 1 further comprising a hydrochloride salt of said compound.

3. The crystal of claim 2 further comprising a hydrate of said compound.

4. The crystal of claim 3 wherein said crystal is of the Type II form.

5. The crystal of claim 3 wherein said crystal is of the Type I form.

6. The crystal of claim 1 wherein said crystal is characterized by a powder X-ray diffraction pattern having at least one powder X-ray diffraction peak with a 2-theta value of about 19.32±0.1°.

7. The crystal of claim 1 wherein said crystal is characterized by a powder X-ray diffraction pattern having a powder X-ray diffraction peak with a 2-theta value of about 19.32±0.1° two-theta and at least one other powder X-ray diffraction peak with a 2-theta value selected from the group consisting of about 6.73±0.1°, 10.77±0.1°, 16.95±0.1° and 29.84±0.1°.

8. The crystal of claim 1 wherein said crystal is characterized by a powder X-ray diffraction pattern having a powder X-ray diffraction peak with a 2-theta value of about 19.32±0.1° and at least one other powder X-ray diffraction peak with a 2-theta value selected from the group consisting of about 6.73±0.1°, 10.77±0.1°, 16.95±0.1°, 22.02±0.1°, 25.42±0.1° and 29.84±0.1°.

9. The crystal of claim 1 having a powder X-ray diffraction pattern that is substantially similar to the powder X-ray diffraction pattern of FIG. 2.

10. The crystal of claim 1 having a powder X-ray diffraction pattern that is substantially similar to the one of the powder X-ray diffraction patterns of FIG. 3.

11. The crystal of claim 1 wherein said crystal is characterized by a TGA thermogram comprising about a 5 percent weight loss between about 30 degrees C. and about 190 degrees C.

12. The crystal of claim 1 wherein said crystal is characterized by a TGA thermogram comprising about a 3 percent weight loss between about 30 degrees C. and about 120 degrees C.

13. The crystal of claim 11 wherein said crystal is further characterized by a TGA thermogram comprising about a 2 percent weight loss between about 120 degrees C. and about 190 degrees C.

14. The crystal of claim 1 wherein said crystal is characterized by having an endothermic transition at about 64° C.

15. The crystal of claim 1 wherein said crystal is characterized by having an endothermic transition at about 133° C.

16. The crystal of claim 1 wherein said crystal is characterized by having an endothermic transition at about 187° C.

17. The crystal of claim 1 wherein said crystal is characterized by having at least one endothermic transition occuring at about 64°, 133°, or 187° C.

18. A method of making the crystal of claim 3, said method comprising the steps of providing the free base form of (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide; adding to said free base an aqueous solution of hydrochloric acid and THF; drying free base solution under controlled condition adequate to produce said crystal.

19. A method of making the crystal of claim 3, said method comprising the steps of providing the free base form of (2R-trans)-6-chloro-5-[[4-[(4-fluorophenyl)methyl]-2,5-dimethyl-1-piperazinyl]carbonyl]-N,N, 1-trimethyl-alpha-oxo-1H-indole-3-acetamide; adding to said free base an aqueous solution of hydrochloric acid, isopropanol, MTBE, and one or more seed crystals; and drying the free base solution under controlled conditions adequate to produce said crystal.

20. A pharmaceutical composition comprising the crystal of claim 1.

21. A pharmaceutical composition comprising the crystal of claim 4.

22. The pharmaceutical composition of claim 21 further comprising one or more pharmaceutically acceptable excipients.
